(19) United States
(12) Patent Application Publication
(10) Pub. No.: US 2023/0397802 A1
(43) Pub. Date: Dec. 14, 2023

(54) ENHANCED GASTROINTESTINAL ENDOSCOPE SUCTION DEVICE

(71) Applicants: Tarun Rustagi, Albuquerque, NM (US); Zain Sobani, Albuquerque, NM (US); Swathi Paleti, Albuquerque, NM (US)

(72) Inventors: Tarun Rustagi, Albuquerque, NM (US); Zain Sobani, Albuquerque, NM (US); Swathi Paleti, Albuquerque, NM (US)

(21) Appl. No.: 18/033,224

(22) PCT Filed: Oct. 20, 2021
(86) PCT No.: PCT/US2021/055792

§ 371 Date: Apr. 21, 2023

Related U.S. Application Data
(60) Provisional application No. 63/104,983, filed on Oct. 23, 2020.
(60) Provisional application No. 63/108,706, filed on Nov. 2, 2020.

(51) Int. Cl. A61B 1/12; A61B 1/015; A61B 1/05; A61B 1/00

(52) U.S. Cl. CPC A61B 1/126; A61B 1/015; A61B 1/05; A61B 1/00135; A61B 1/00066; A61B 1/2736

(57) ABSTRACT

An endoscope device comprises an operating unit comprising one or more controls for operating the endoscope device and an elongate insertion member coupled to the operating unit. The insertion member comprises an elongate endoscope shaft extending from a shaft proximal end proximate to the operating unit to a shaft distal end, a digital imaging device coupled to the shaft distal end of the endoscope shaft, and an elongate suction channel with a channel distal end that is positioned proximate to the shaft distal end so that suction applied to the suction channel can clear debris from a visual field of the digital imaging device.

### PRIORITY APPLICATIONS

This application claims the benefit of priority to U.S. Provisional Patent Application Ser. No. 63/104,983, filed Oct. 23, 2020 and claims the benefit of priority to U.S. Provisional Patent Application Ser. No. 63/108,706, filed Nov. 2, 2020, the contents of both which are incorporated herein by reference in their entireties.

### BACKGROUND

When performing upper gastrointestinal endoscopy, it is not infrequent to encounter blood clots, food, and residual gastric content. This material impairs the visual field during the procedure, especially in cases of gastrointestinal bleeding and food impaction. These materials are often difficult to clear with conventional endoscopes. Even therapeutic gastroscopes, which have suction channels that are wider than conventional endoscopes (e.g., 3.7 millimeters (mm) for a single-channel therapeutic gastroscope), are often unable to adequately suction blood, blood clots, and food residue to clear the upper gastrointestinal tract for adequate endoscopic visualization.

### SUMMARY

According to one aspect of the present disclosure, an endoscope device is described. In an example, the endoscope device comprises an operating unit comprising one or more controls for operating the endoscope device and an elongate insertion member coupled to the operating unit. In an example, the insertion member comprises an elongate endoscope shaft extending from a shaft proximal end proximate to the operating unit to a shaft distal end, a digital imaging device coupled to the shaft distal end of the endoscope shaft, and an elongate suction channel with a channel distal end that is positioned proximate to the shaft distal end so that suction applied to the suction channel can clear debris from a visual field of the digital imaging device.

According to another aspect of the present disclosure, an accessory for an endoscope is described. In an example, the accessory includes an external elongate suction channel configured for coupling to an elongated endoscope shaft of an insertion member of the endoscope, wherein the suction channel is configured so that when the suction channel is coupled to the endoscope shaft, a channel distal end will be positioned proximate to a shaft distal end of the endoscope shaft so that suction applied to the suction channel can clear debris from an area around the shaft distal end.

According to another aspect of the present disclosure, an endoscope device is described. In an example, the endoscope device comprises an operating unit comprising one or more controls for operating the endoscope device and an elongate insertion member coupled to the operating unit. In an example, the insertion member comprises an elongate endoscope shaft extending from a shaft proximal end proximate to the operating unit to a shaft distal end, a digital imaging device coupled to the shaft distal end of the endoscope shaft, and a plurality of elongate suction channels each with a channel distal end that is positioned proximate to the shaft distal end so that suction applied to each of the plurality of elongate suction channels can clear debris from a visual field of the digital imaging device.

According to another aspect of the present disclosure, an accessory for an endoscope is described. In an example, the accessory includes a plurality of external elongate suction channels that are each configured for coupling to an elongated endoscope shaft of an insertion member of the endoscope, wherein each of the plurality of suction channels is configured so that when the suction channel is coupled to the endoscope shaft, a channel distal end of each of the plurality of suction channels will be positioned proximate to a shaft distal end of the endoscope shaft so that suction applied to the plurality of suction channels can clear debris from an area around the shaft distal end.

### BRIEF DESCRIPTION OF THE DRAWINGS

The drawings illustrate generally, by way of example, but not by way of limitation, various embodiments discussed in the present document.

FIG. 1 is a side view of a first example endoscope device, in accordance with the present disclosure.

FIG. 2 is an end view of the distal end of the first example endoscope device shown in FIG. 1.

FIG. 3 is a side view of a second example endoscope device, in accordance with the present disclosure.

FIG. 4 is an end view of the distal end of the second example endoscope device shown in FIG. 3.

FIG. 5 is a side view of a third example endoscope device, in accordance with the present disclosure.

FIGS. 6A-6B are end views of the distal end of the third example endoscope device shown in FIG. 5.

FIG. 7 is a side view of a fourth example endoscope device, in accordance with the present disclosure.

FIG. 8 is an end view of the distal end of the fourth example endoscope device shown in FIG. 7.

FIG. 9 is a side view of a fourth example endoscope device, in accordance with the present disclosure.

FIG. 10 is a perspective view of an insertion member portion of the fourth example endoscope device of FIG. 9.

FIG. 11 is an end view of the distal end of the fourth example endoscope device shown in FIGS. 9 and 10.

FIG. 12A is a schematic view of a first example suction source system for supplying suction to the fourth example endoscope device of FIGS. 9-11.

FIG. 12B is a schematic view of a second example suction source system for supplying suction to the fourth example endoscope device of FIGS. 9-11.

FIG. 13 is a perspective view of an insertion member portion of a fifth example endoscope device, in accordance with the present disclosure.

FIG. 14 is an end view of the distal end of the insertion member portion of the fifth example endoscope device of FIG. 13.

FIG. 15 is a perspective view of an insertion member portion of a sixth example endoscope device, in accordance with the present disclosure.

FIG. 16 is an end view of the distal end of the insertion member portion of the sixth example endoscope device of FIG. 15

FIG. 17 is a side view of a seventh example endoscope device, in accordance with the present disclosure.

FIG. 18 is a perspective view of the insertion member portion of the seventh example endoscope device of FIG. 17.

FIG. 19 is an end view of the distal end of the seventh example endoscope device of FIG. 17.

FIG. 20 is a schematic view of an example suction source system for supplying suction to the seventh example endoscope device of FIG. 17.

### DETAILED DESCRIPTION

The following detailed description describes endoscopic devices with enhanced suction for use during an upper gastrointestinal endoscopy procedure. It also describes accessories for use with an endoscope that provide for enhanced suction during upper gastrointestinal endoscopy procedures. The present disclosure also describes methods of performing gastrointestinal endoscopy with enhanced suction to more effectively clear blood, blood products, blood clots, food particles, and residual gastric content. The endoscopic devices or the endoscopic accessories include one or more suction channels that are external to and connected to the endoscope so that the one or more suction channels can be directed to target areas using the endoscope.

The following specification includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The example embodiments may be combined, other embodiments may be utilized, or structural, and logical changes may be made without departing from the scope of the present invention. While the disclosed subject matter will be described in conjunction with the enumerated claims, it will be understood that the exemplified subject matter is not intended to limit the claims to the disclosed subject matter. The following detailed description is, therefore, not to be taken in a limiting sense, and the scope of the present invention is defined by the appended claims and their equivalents.

References in the specification to “one embodiment”, “an embodiment,” “an example embodiment,” etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a concentration range of “about 0.1% to about 5%” should be interpreted to include not only the explicitly recited concentration of about 0.1 wt. % to about 5 wt. %, but also the individual concentrations (e.g., 1%, 2%, 3%, and 4%) and the sub-ranges (e.g., 0.1% to 0.5%, 1.1% to 2.2%, and 3.3% to 4.4%) within the indicated range. The statement “about X to Y” has the same meaning as “about X to about Y,″” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. Unless indicated otherwise, the statement “at least one of” when referring to a listed group is used to mean one or any combination of two or more of the members of the group. For example, the statement “at least one of A, B, and C” can have the same meaning as “A; B; C; A and B; A and C; B and C; or A, B, and C,” or the statement “at least one of D, E, F, and G” can have the same meaning as “D; E; F; G; D and E; D and F; D and G; E and F; E and G: F and G; D, E, and F; D, E, and G; D, F, and G; E, F, and G; or D, E, F, and G.” A comma can be used as a delimiter or digit group separator to the left or right of a decimal mark; for example, “0.000, 1″” is equivalent to “0.0001.”

In the methods described herein, the steps can be carried out in any order without departing from the principles of the invention, except when a temporal or operational sequence is explicitly recited. Furthermore, specified steps can be carried out concurrently unless explicit language recites that they be carried out separately. For example, a recited act of doing X and a recited act of doing Y can be conducted simultaneously within a single operation, and the resulting process will fall within the literal scope of the process. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E (including with one or more steps being performed concurrent with step A or Step E), and that the sequence still falls within the literal scope of the claimed process. A given step or sub-set of steps can also be repeated.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, within 1%, within 0.5%, within 0.1%, within 0.05%, within 0.01%, within 0.005%, or within 0.001% of a stated value or of a stated limit of a range and includes the exact stated value or range.

The term “substantially” as used herein refers to a majority of, or mostly, such as at least about 50%, 60%, 70%, 80%, 90%, 95%, 96%, 97%, 98%, 99%, 99.5%, 99.9%, 99.99%, or at least about 99.999% or more, or 100%.

In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

FIG. 1 is an overall view of an example endoscope device 10 that can be used for the examination of a cavity within a patient. For example, the endoscope device 10 can be a gastroscope used for examining the gastrointestinal tract of a human patient, such as the esophagus stomach, and small intestine. The endoscope device 10 can include an operating unit 12 and an insertion member 14 coupled to the operating unit 12. The operating unit 12 can include one or more controls 16 that allow a user, such as a doctor, to operate the endoscope device 10. A tool inlet channel 18 can be included on the operating unit 12, which can provide for an inlet through which a tool can be inserted so that the tool will be inserted into the insertion member 14.

The insertion member 14 can include a proximal end 20 coupled to the operating unit 12, a distal tip 22, and an elongated shaft 24 extending from the proximal end 20 to the distal tip 22. A digital imaging device 26 can be mounted at the distal tip 22 of the insertion member 14 to capture images or video of the view at the distal tip 22 (e.g., from within the patient cavity being examined). In an example, the digital imaging device 26 can comprise a charge coupled device (CCD) light sensor.

In an example, the endoscope device 10 comprises a low-profile endoscope. As used herein, the term “low-profile endoscope” refers to an endoscope with an outer diameter of the insertion portion of the endoscope, e.g., the endoscope shaft 24, that is smaller than that of a conventional endoscope (which is typically at least about 9 mm and often is as much as 12 mm). In an example, the term “low-profile endoscope” refers to an endoscope with an insertion member outer size of 9 mm or less, such as 8.5 mm or less, for example 8 mm or less, such as 7.5 mm or less, for example 7 mm or less, such as 6.5 mm or less, for example 6 mm or less, such as 5.5. mm or less, for example 5 mm or less, such as 4.5 mm or less, for example 4 mm or less. In an example, the low-profile endoscope shaft 24 has a size of from about 4 mm to about 9 mm, such as about 5 mm. An example of a low-profile endoscope that could be used as part of the endoscope device 10 of the present disclosure is the Olympus EVIS EXERA III GIF-XP190N sold by Olympus Medical.

In an example, a portion of the insertion member 14 can be deflectable for better maneuverability of the insertion member 14 through the patient cavity. For example, the elongated shaft 24 can be configured so that the distal tip 22 can be deflected in one or more directions for maneuvering through tight orifices or sharp bends in the patient's body cavity. Deflection of the distal tip 22 or other portions of the insertion member 14 can also be employed to allow the user performing the examination a better angle of view toward a particular portion of the body cavity. Deflection of the distal tip 22 or any other portion of the insertion member 14 can be controlled by the controls 16 on the operating unit 12.

An umbilical cable 28 can be coupled to the operating unit 12, for example to provide one or more of an electrical connection to a power source (not shown) and for a communication cable for carrying a video signal of the images or video captured by the digital imaging device 26. Other optional functionalities can also be supplied through the umbilical cable 28, such as an air source (not shown) for pumping air into and through the insertion member 14 (e.g., for expanding the cavity with air to provide for a better view of the cavity), or a water source (not shown) for pumping water into and through the insertion member 14, e.g., to clear an obstruction away from the distal tip of the insertion member 14.

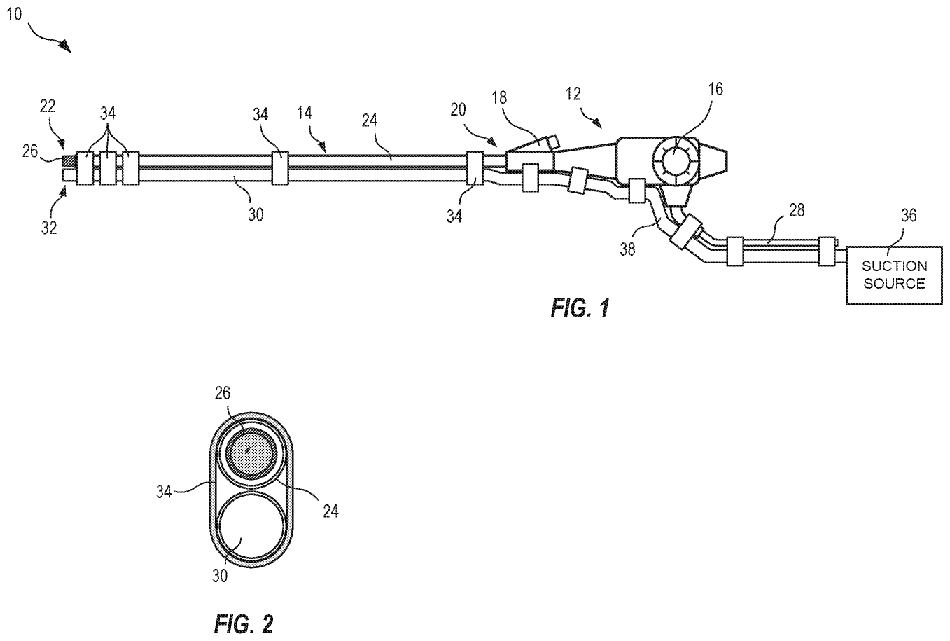

The endoscope device 10 also includes an elongated suction channel 30 with a distal end 32. The suction channel 30 extends in generally the same direction as the shaft 24 of the insertion member 14. For example, in the example endoscope device 10 of FIG. 1, the suction channel 30 is positioned adjacent to the shaft 24 so that the shaft 24 and the suction channel 30 are effectively running parallel to one another. The suction channel 30 is positioned so that its distal end 32 is located proximate to the distal tip 22 of the endoscope shaft 24 so that suction that is provided through the suction channel 30 can clear away debris, such as blood, blood clots, food particles, and residual gastric content, so that the debris is clear of the visual field of the digital imaging device 26. FIG. 2 shows an end view of the distal end of the endoscope device 10, e.g., looking toward the digital imaging device 26, the distal tip 22 of the endoscope shaft 24, and the distal end 32 of the suction channel 30.

As noted above, conventional endoscopes that can be used as a gastroscope have a relatively small suction channel size, such as the Olympus EVIS EXERA III GIF-HQ190 sold by Olympus Medical, which has an inner channel size of about 2.8 millimeters (mm). Channels are typically larger on therapeutic endoscopes, such as the Olympus EVIS EXERA III GIF-1TH190 single-channel therapeutic gastroscope, which has an inner channel size of about 3.7 mm. While these channels are wider than conventional endoscopes, these channel sizes are still the main limitation to the suction capability of the gastroscope. It is still quite common for these gastroscopes to be unable to fully clear the debris associated with an upper gastrointestinal endoscopy.

In an example, the suction channel 30 of the endoscope device 10 of the present disclosure has a relatively larger size than the suction channels of conventional gastroscopes. In an example, the suction channel 30 has an inner diameter of at least about 4 mm, for example, at least about 4.5 mm, such as at least 5 mm. In the example of a 5 mm inner diameter suction channel 30, the cross-sectional area of the suction channel 30 is about 218% larger than that of the example conventional gastroscope and about 82.6%% larger than that of the example therapeutic gastroscope. And since the suction capacity is proportional to the cross-sectional area of the opening of the suction channel 30, this results in substantially higher suction capability for the endoscope device 10 of the present disclosure as compared to the more conventional gastroscopes. In an example, the suction channel 30 has an internal cross-sectional area of at least about 15 square millimeters (mm²), such as at least about 16 mm², for example at least about 16.5 mm²′ such as at least about 17 mm², for example at least about 17.5 mm², such as at least about 18 mm, for example at least about 18.5 mm², such as at least about 19 mm², for example at least about 19.5 mm², such as at least about 19.6 mm², for example at least about 20 mm², such as at least about 20.5 mm², for example at least about 21 mm², such as at least about 22 mm², for example at least about 23 mm², such as at least about 24 mm², for example at least about 25 mm².

In an example, the suction channel 30 is coupled at least to the endoscope shaft 24, such as the shaft of a low-profile endoscope (such as Olympus EVIS EXERA III GIF-XP190N). In the example shown in FIG. 1, the suction channel 30 is coupled to the endoscope shaft 24 with one or more coupling devices or structures, such as medical tape 34 that couples the suction channel 30 to the endoscope shaft 24 at one or more locations along the shaft 24, such as at 3 or more locations along the shaft 24. However, those having skill in the art will appreciate that other means of coupling the suction channel 30 to the endoscope shaft 24 can be used without varying from the scope of the present disclosure, including, but not limited to clips, straps, adhesive, or plastic welding.

In an example, at least one connection between the suction channel 30 and the endoscope shaft 24, e.g., with the tape 34 or another coupling structure, is located proximate to the distal tip 22 of the shaft 24 and the distal end 32 of the suction channel 30 so that when the distal tip 22 of the shaft 24 is deflected while maneuvering the insertion member 14, the distal end 32 of the suction channel 30 will also be deflected in the same direction and with approximately the same amount of deflection. The matching or substantially matching deflection of the suction channel 30 with that of the endoscope shaft 24 can ensure that direction of suction into the suction channel 30 is pointed generally or substantially in the same direction as the digital imaging device 26 so that the suction will clear the visual field of the digital imaging device 26. In an example, the suction channel 30 is coupled closely to the shaft 24 so that the shaft 24 and the suction channel 30 can be more easily inserted into the patient cavity, e.g., so that the endoscope shaft 24 and the suction channel 30 together act as the insertion member of the endoscope device 10.

Suction can be supplied to the suction channel 30 by a suction source 36 via a suction supply line 38. In an example, the suction channel 30 and the suction supply line 38 can be configured to receive suction energy from a suction source 36 comprising any of the many devices used in the medical field for providing suction. In an example, the suction source 36 is a standard wall suction, e.g., that is supplied to operating theaters in most hospitals. In another example, the suction source 26 can be an enhanced suction device, which may or may not be portable, that is configured to supply a larger suction force compared to standard wall suction devices. An example of such an enhanced suction device that can be used as the suction source 36 includes, but is not limited to, the system sold under the NEPTUNE 3 Waste Management System trade name by Stryker Corp., Kalamazoo, MI, USA. In an example, the suction supply line 38 is separate from the umbilical cable 28, as shown in FIG. 1. In an example, shown in FIG. 1, the suction supply line 38 is the same tube that defines the suction channel 30. In other words, in an example, the suction channel 30 and the suction supply line 38 can be the same structure, e.g., the same tube. However, those having ordinary skill in the art will be able to readily design a suction feed system where the suction supply line 38 is separate from the suction channel 30 and in fluid communication with the suction channel 30.

In an example, suction through the suction channel 30 is controllable. For example, the user of the endoscope device 10 may be able to turn on and turn off suction through the suction channel 30. In other examples, the amount of suction force that is drawn through the suction channel 30 can be adjustable and controllable, such as with the one or more controls 16 or with some other controlling mechanism.

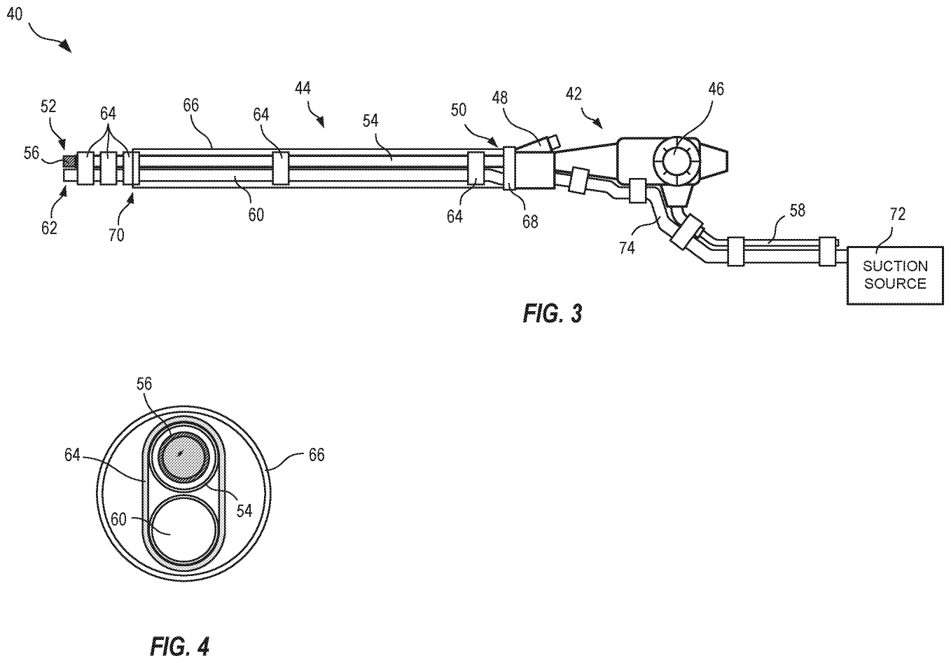

FIGS. 3 and 4 show another example of an endoscope device 40 that can be used for examination of a cavity within a patient, such as a gastroscope. The endoscope device 40 in FIG. 3 includes many of the same structures as the endoscope device 10 of FIGS. 1 and 2, which can be similar or even identical to the structures described above with respect to the endoscope device 10. For example, the endoscope device 40 can include: an operating unit 42; an insertion member 44 coupled to the operating unit 42; one or more controls 46 so that a user can operate the endoscope device 40; a tool inlet channel 48; an elongated shaft 54 that extends between a proximal end 50 coupled to the operating unit 42 and a distal tip 52; a digital imaging device 56 at the distal tip 52 of the shaft 54; an umbilical cable 58 coupled to the operating unit 42 to provide specified functionality to the endoscope device 40; and an elongated suction channel 60 that is coupled to the endoscope shaft 54, such as with medical tape 64, at least proximate to the distal tip 52 so that a distal end 62 of the suction channel 60 can deflect with the distal tip 52.

In addition to these aspects, the insertion member 44 of the example endoscope device 40 in FIGS. 3 and 4 can include an optional overtube 66 through which the endoscope shaft 54 and the suction channel 60 can be inserted. In an example, the overtube 66 can make insertion of the insertion member 44 into the patient cavity easier, e.g., because the outer surface of the overtube 66 is smoother than the outside profile of the endoscope shaft 54 and the suction channel 60. The overtube 66 can have a size that is large enough to cover both the endoscope shaft 54 and the suction channel 60, but not so large that it cannot be inserted into the patient cavity. In an example, the overtube 66 has an inner diameter of from about 10 mm to about 20 mm, such as from about 15 mm to about 17.5 mm, for example about 16.7 mm.

In an example, the overtube 66 can be coupled to the operating unit 42, such as with a fixture 68. In an example, the overtube 66 is a standard overtube, such as those sold under the GUARDUS trademark by Steris Endoscopy, Mentor, OH, USA.

As shown in FIG. 3, the overtube 66 can be configured so that the distal tip 52 of the endoscope shaft 54 and the distal end 62 of the suction channel 60 can be extended out beyond a distal end 70 of the overtube 66. In another example, the overtube 66 can be configured so that the endoscope shaft 54 and the suction channel 60 can be retracted inside of the overtube 66 (e.g., so that the distal tip 52 of the endoscope shaft 54 and the distal end 62 of the suction channel 60 are behind or proximal to the distal end 70 of the overtube 66) during insertion into the patient cavity, and once the distal end 70 of the overtube 66 reaches the target site, the endoscope shaft 54 and the suction channel 60 can be extended out past the distal end 70 of the overtube 66 to reach the target site. FIG. 4 is an end view of the distal end of the endoscope device 40 of FIG. 3, e.g., showing a view looking toward the digital imaging device 56, the distal tip 52 of the endoscope shaft 54, the distal end 62 of the suction channel 60, and the distal end 70 of the overtube 66.

Similar to the endoscope device 10 of FIG. 1, suction can be supplied to the suction channel 60 of the endoscope device 40 by a suction source 72 via a suction supply line 74. The suction source 72 and the suction supply line 74 can be similar or identical to the suction source 36 and the suction supply line 38 described above. In an example, the suction supply line 74 passes through a portion of the operating unit 42 and/or through the fixture 68 that couples the overtube 66 to the operating unit 42. The suction supply line 74 can be the same structure, e.g., the same tube, as the suction channel 60 or it can be a separate channel that is in fluid communication with the suction channel 60.

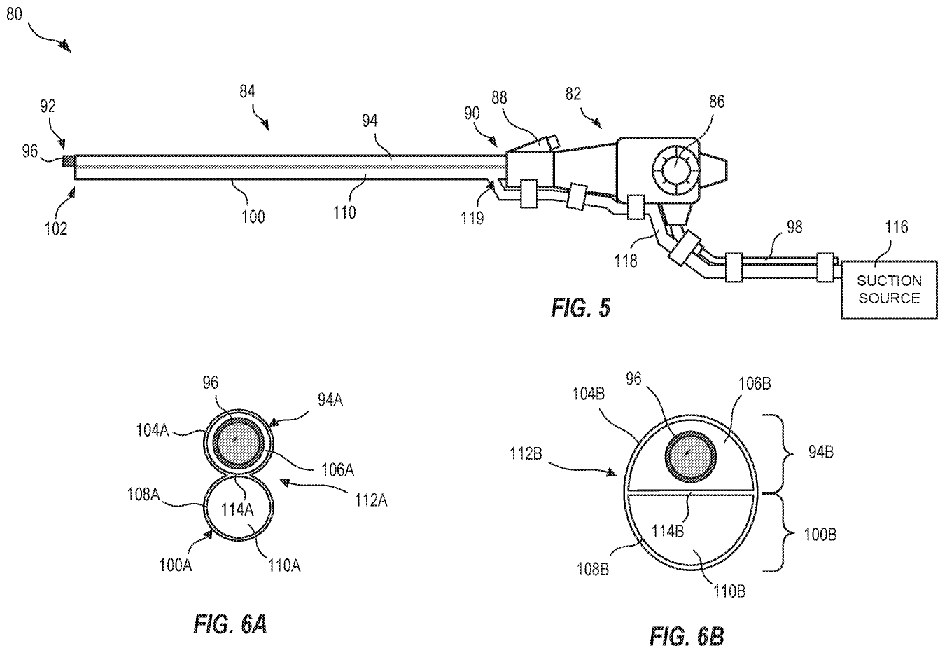

FIG. 5 shows yet another example of an endoscope device 80 that can be used for examination of a cavity within a patient, such as a gastroscope. The endoscope device 80 in FIG. 5 includes many of the same structures as the endoscope devices 10 and 40, which can be similar or even identical to the structures described above with respect to the endoscope devices 10 and 40. For example, the endoscope device 80 can include: an operating unit 82; an insertion member 84 coupled to the operating unit 82; one or more controls 86 so that a user can operate the endoscope device 80; a tool inlet channel 88; an elongated endoscope shaft 94 that extends between a proximal end 90 coupled to the operating unit 82 and a distal tip 92; a digital imaging device 96 at the distal tip 92 of the shaft 94; and an umbilical cable 98 coupled to the operating unit 82 to provide specified functionality to the endoscope device 80.

The example endoscope device 80 of FIG. 5 also includes an elongated suction channel 100, which serves the same purpose to that of the suction channels 30 and 60 in the endoscope devices 10 and 40, i.e., to provide suction at a distal end 102 of the suction channel 100 that is proximate to the distal tip 92 of the endoscope shaft 94 that is sufficient to clear debris commonly associated with gastrointestinal endoscopy, such as food particles, blood, blood clots, and other residual gastric content. Also similar to the endoscope channels 30 and 60, the endoscope channel 100 of the endoscope device 80 runs parallel to the endoscope shaft 94, e.g., so that the distal end 102 of the suction channel 100 will be aligned in substantially the same direction as the distal tip 92 of the endoscope shaft 94. However, instead of being coupled to the endoscope shaft 94 with a coupling device or structure, such as the medical tape 34, 64 used in the endoscope devices 10 and 40, the outer walls of the endoscope shaft 94 and the suction channel 100 are formed from a single unitary or substantially unitary structure. As used herein, the term “unitary or substantially unitary structure,” when referring to the endoscope shaft 94 and the suction channel 100 of the example endoscope device 80, refers to a structure where the endoscope shaft 94 and the suction channel 100 are coupled together along their entire length or substantially their entire length so that they essentially are a single piece. In particular, the endoscope shaft 94 and the suction channel 100 are unitary or substantially unitary proximate to the distal tip 92 of the shaft 94 and the distal end 102 of the suction channel 100 so that when the distal tip 92 is deflected by a user, the distal end 102 of the suction channel 100 will also be deflected in the same direction and with approximately the same amount of deflection as the deflection of the distal tip 92 of the endoscope shaft 94.

FIGS. 6A and 6B show end views of two alternative configurations of the unitary or substantially unitary endoscope shaft 94 and suction channel 100 for the endoscope device 80. In FIG. 6A, a first example of the endoscope shaft 94A and a first example of the suction channel 100A are each formed essentially from an individual tube (such as typical plastic tubing), e.g., with the endoscope shaft 94A being defined by a first wall 104A surrounding a first cylindrical lumen 106A and with the suction channel 100A being defined by a second wall 108A surrounding a second cylindrical lumen 110A that is separate from the shaft's lumen 106A. In the example configuration of FIG. 6, the separate tube structures of the endoscope shaft 94 and the suction channel 100 are joined together, such as by plastic welding, direct fastening, or an adhesive, to form essentially a single overall sheath 112A. In the example shown in FIG. 6A, the tube wall 104A that defines the endoscope shaft 94A is fused with the tube wall 108A that defines the suction channel 100A such that the portions of the walls 104A, 106A that are located between the two lumens 106A and 110A combine to form a fused wall 114A between the lumen 106A of the endoscope shaft 94A and the lumen 110A of the suction channel 100A.

FIG. 6B shows a second example configuration of a unitary or substantially unitary structure for the endoscope shaft 94 and the suction channel 100. In the example of FIG. 6B, the endoscope shaft 94B and the suction channel 100B are sections of the same cylindrical tube 112B that has been subdivided by an interior wall 114B to separate what would be the large cylindrical lumen of the tube 112B into a semi-cylindrical first lumen 106B for the endoscope shaft portion 94B (e.g., through which the digital imaging device 96 is fed) and a semi-cylindrical second lumen 110B for the suction channel portion 100B (e.g., through which the suction flows and through which the debris passes as it is cleared away from the distal tip 92 of the endoscope shaft portion 94B). Those having skill in the art will appreciate that there can be many other configurations of a unitary or substantially unitary endoscope shaft 94 and suction channel 100 for the example endoscope device 80 of FIG. 5.

Similar to the endoscope devices 10 and 40, suction can be supplied to the suction channel 100 of the endoscope device 80 by a suction source 116 via a suction supply line 118. The suction source 116 and the suction supply line 118 can be similar or identical to the suction source 36, 72 and the suction supply line 38, 74 described above. The suction supply line 118 can be the same structure, e.g., the same tube, as the suction channel 100 or it can be a separate channel that is in fluid communication with the suction channel 100. In an example, shown in FIG. 5, the suction supply line 118 is in fluid communication with the lumen 110 of the suction channel 100, such as through a port 119 in the suction channel 100.

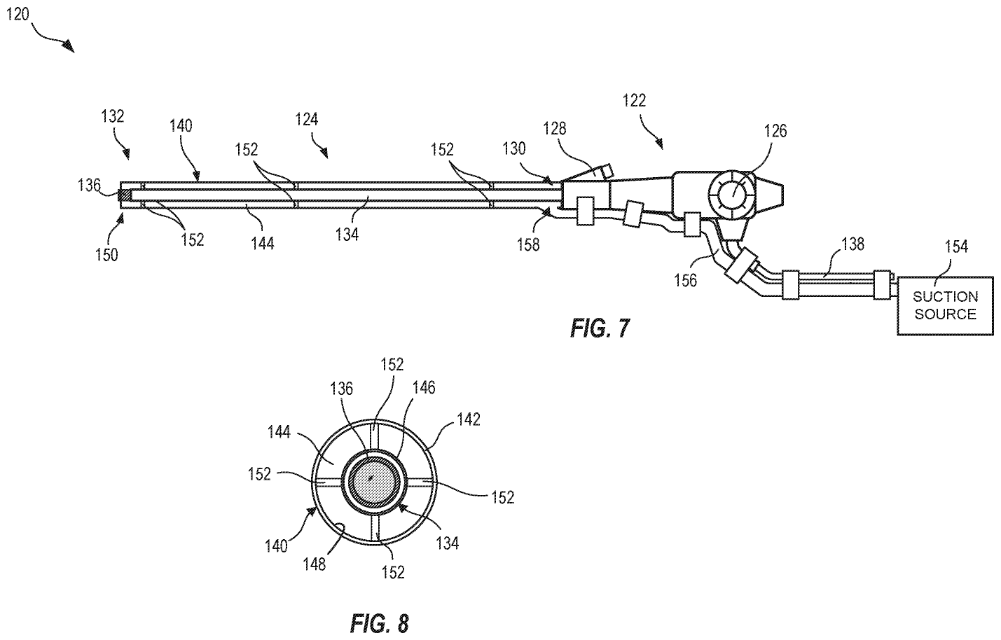

FIGS. 7 and 8 show still another example of an endoscope device 120 that can be used for examination of a cavity within a patient, such as a gastroscope. The endoscope device 120 includes many of the same structures as the endoscope devices 10, 40, and 80, which can be similar or even identical to the structures described above with respect to the endoscope devices 10, 40, and 80. For example, the endoscope device 120 can include: an operating unit 122; an insertion member 124 coupled to the operating unit 122; one or more controls 126 so that a user can operate the endoscope device 120; a tool inlet channel 128; an elongated endoscope shaft 134 that extends between a proximal end 130 coupled to the operating unit 132 and a distal tip 132; a digital imaging device 136 at the distal tip 132 of the shaft 134; and an umbilical cable 138 coupled to the operating unit 122 to provide specified functionality to the endoscope device 120.

The example endoscope device 120 of FIGS. 7 and 8 also includes an elongated suction channel 140, which serves a similar purpose to that of the suction channels 30, 60, and 100 in the endoscope devices 10, 40, and 80, i.e., to provide suction at the distal tip 132 that is sufficient to clear debris commonly associated with gastrointestinal endoscopy, such as food particles, blood, blood clots, and other residual gastric content. However, the configuration of the suction channel 140 of the endoscope device 120 is different from that of the suction channels 30, 60, and 100. Instead of being a channel that runs alongside the endoscope shaft 134, the suction channel 140 is a sheath-like structure that fits over and around the endoscope shaft 134, such as a concentric sheath wall 142 that surrounds the endoscope shaft 134 as shown in FIGS. 7 and 8, so that the endoscope shaft 134 is located within a lumen 144 of the suction channel 140. The location of the endoscope shaft 134 within the lumen 144 of the suction channel 140 is also shown in the end view of FIG. 8. In this way, the suction that passes through the suction channel 140 would be located in the annular space within the lumen 144 located between an outer surface 146 of the endoscope shaft 94 and an inner surface 148 of the sheath wall 142 of the suction channel 140.

In an example, the sheath wall 142 of the suction channel 140 is configured so that a distal end 150 of the sheath wall 142 will be located proximate to the digital imaging device 136 at the distal tip 132 of the endoscope shaft 134. As described above with respect to the suction channel 30, this positioning can ensure that that the suction into the suction channel 140 can clear away debris from the visual field of the digital imaging device 136.

In an example, the sheath wall 142 of the suction channel 140 can be deflectable along with the deflectability of the endoscope shaft 134, e.g., so that if the distal tip 132 of the endoscope shaft 134 is deflected while maneuvering the shaft 134 through the patient cavity, the distal end 150 of the sheath wall 142 will also be deflected in the same direction and generally the same amount as the distal tip 132. As noted above, this can help ensure that the direction of suction into the suction channel 140 will continue to clear debris from the visual field of the digital imaging device 136.

The size of the sheath wall 142 of the suction channel 140 can be selected so that the annular space of the lumen 144 between the endoscope shaft 134 and the suction channel sheath wall 142 is sized to achieve a specified suction capacity through the suction channel 140. As noted above, the suction capacity through a suction channel is proportional to its cross-sectional area, which in this case is the cross-sectional area of the annular space of the lumen 144. In an example, the endoscope shaft 134 has an outer diameter of about 4 mm to about 6 mm, such as about 5 mm, and the sheath wall 142 of the suction channel 140 has an inner diameter of from about 6 mm to about 8 mm, such as from about 6.5 mm to about 7.5 mm. In an example, the cross-sectional area of the annular lumen 144 (e.g., the cross-sectional area of the space between the outer surface 146 of the endoscope shaft 134 and the inner surface 148 of the suction channel sheath wall 142 is at least about 15 square millimeters (mm²), such as at least about 16 mm², for example at least about 16.5 mm², such as at least about 17 mm², for example at least about 17.5 mm², such as at least about 18 mm², for example at least about 18.5 mm², such as at least about 19 mm², for example at least about 19.5 mm², such as at least about 19.6 mm², for example at least about 20 mm², such as at least about 20.5 mm², for example at least about 21 mm², such as at least about 22 mm², for example at least about 23 mm², such as at least about 24 mm², for example at least about 25 mm².

In an example, the suction channel sheath wall 142 can be coupled to the endoscope shaft 134 at various intermittent points along the length of the shaft 94, such as with connector pins 152. The connector pins 152 can ensure that deflection of the endoscope shaft 134 will result in a corresponding deflection of the suction channel sheath 142. In another example, the connector pins 152 can ensure a specified spacing between the endoscope shaft 134 and the sheath wall 142 of the suction channel 140, e.g., so that the cross-sectional area of the lumen 144 through which the suction travels is large enough to achieve a desired suction capacity.

Similar to the endoscope devices 10, 40, and 80, suction can be supplied to the suction channel 140 of the endoscope device 120 by a suction source 154 via a suction supply line 156. The suction source 154 and the suction supply line 156 can be similar or identical to the suction sources 36, 72, 116 and the suction supply lines 38, 74, 118 described above. In an example, the suction supply line 156 is a separate channel from the suction channel 140, wherein the suction supply line 156 is in fluid communication with the lumen 144 of the suction channel 140. In an example, shown in FIG. 7, the suction supply line 146 is in fluid communication with the lumen 144 through a port 158 in the suction channel 100.

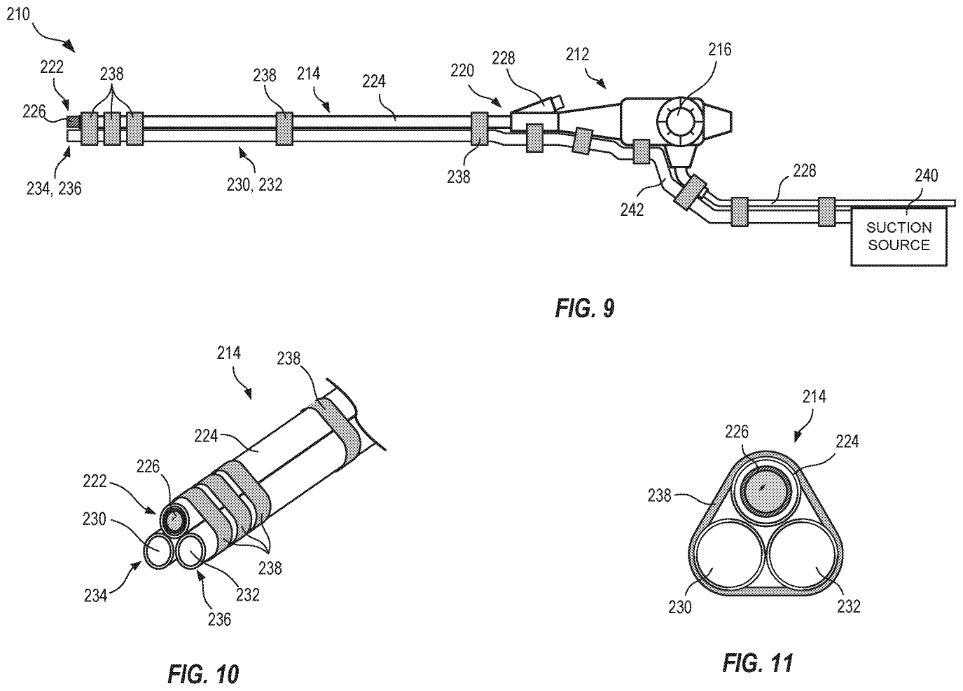

FIGS. 9 and 10 show yet another example of an endoscope device 210 that can be used for the examination of a cavity within a patient, such as a gastroscope for examining the gastrointestinal tract of a human patient, such as the esophagus stomach, and small intestine. The endoscope device 210 can include many of the same structures as the endoscope devices 10, 40, 80, 120, which can be similar or even identical to the structures described above with respect to the endoscope devices 10, 40, 80, and 120. For example, the endoscope device 210 can include an operating unit 212, an insertion member 214 coupled to the operating unit 212, one or more controls 216 that allow a user, such as a doctor, to operate the endoscope device 210, a tool inlet channel 218, an elongated shaft 224 (also referred to as “the endoscope shaft 224”) that extends between a proximal end 220 coupled to the operating unit 212 and a distal tip 222, and a digital imaging device 226 mounted at the distal tip 222 of the elongated shaft 224 to capture images or video of the view at the distal tip 222 (e.g., from within the patient cavity being examined). In an example, the digital imaging device 226 can comprise a charge coupled device (CCD) light sensor.

In an example, the endoscope device 210 comprises a low-profile endoscope, as described above with respect to the endoscope device 10, for example an endoscope with an insertion member outer size of 9 mm or less, such as 8.5 mm or less, for example 8 mm or less, such as 7.5 mm or less, for example 7 mm or less, such as 6.5 mm or less, for example 6 mm or less, such as 5.5. mm or less, for example 5 mm or less, such as 4.5 mm or less, for example 4 mm or less. In an example, the low-profile endoscope shaft 224 has a size of from about 4 mm to about 9 mm, such as about 5 mm. An example of a low-profile endoscope that could be used as part of the endoscope device 10 of the present disclosure is the Olympus EVIS EXERA III GIF-XP190N sold by Olympus Medical.

In an example, a portion of the insertion member 214 can be deflectable for better maneuverability of the insertion member 214 through the patient cavity. For example, the elongated shaft 224 can be configured so that the distal tip 222 can be deflected in one or more directions for maneuvering through tight orifices or sharp bends in the patient's body cavity. Deflection of the distal tip 222 or other portions of the insertion member 214 can also be employed to allow the user performing the examination a better angle of view toward a particular portion of the body cavity with the digital imaging device 226. Deflection of the distal tip 222 or any other portion of the insertion member 214 can be controlled by the controls 216 on the operating unit 212.

An umbilical cable 228 can be coupled to the operating unit 212, for example to provide one or more of an electrical connection to a power source (not shown) and for a communication cable for carrying a video signal of the images or video captured by the digital imaging device 226. Other optional functionalities can also be supplied through the umbilical cable 228, such as an air source (not shown) for pumping air into and through the insertion member 214 (e.g., for expanding the cavity with air to provide for a better view of the cavity), or a water source (not shown) for pumping water into and through the insertion member 214, e.g., to clear an obstruction away from the distal tip of the insertion member 214.

The endoscope device 10 also includes a pair of elongated suction channels 230, 232 that each extend in generally the same direction as the shaft 224 of the insertion member 214. For example, in the example endoscope device 210 of FIG. 9, the suction channels 230, 232 are positioned adjacent to the shaft 224 so that the shaft 224 and the suction channels 230, 232 are effectively running parallel to one another. A first of the pair of suction channels, e.g., the suction channel 230, extends to a distal end 234, and a second of the pair of suction channels, e.g., the suction channel 232, extends to a distal end 236. The suction channels 230, 232 are positioned so that the distal ends 234, 236 are located proximate to the distal tip 222 of the endoscope shaft 224 so that suction that is provided through the suction channels 230, 232 can clear away debris, such as blood, blood clots, food particles, and residual gastric content, so that the debris is clear of the visual field of the digital imaging device 226. FIG. 10 shows a perspective view of the insertion member portion 214 of the endoscope device 210, e.g., of the endoscope shaft 224 and the suction channels 230, 232 without the operating unit 212. FIG. 11 shows an end view of the distal end of the endoscope device 210, e.g., looking toward the digital imaging device 226, the distal tip 222 of the endoscope shaft 224, and the distal ends 234, 236 of the suction channels 230, 232.

As noted above, conventional endoscopes that can be used as a gastroscope have a relatively small suction channel size, such as the Olympus EVIS EXERA III GIF-HQ190 sold by Olympus Medical, which has an inner channel size of about 2.8 millimeters (mm). Channels are typically larger on therapeutic endoscopes, such as the Olympus EVIS EXERA III GIF-1TH190 single-channel therapeutic gastroscope, which has an inner channel size of about 3.7 mm. While these channels are wider than conventional endoscopes, these channel sizes are still the main limitation to the suction capability of the gastroscope. It is still quite common for these gastroscopes to be unable to fully clear the debris associated with an upper gastrointestinal endoscopy.

In an example, the suction channels 230, 232 of the endoscope device 210 of the present disclosure have a combined size that is relatively larger size than the suction channel of conventional gastroscopes. In an example, each of the suction channels 230, 232 has an inner diameter of at least about 3 mm, for example at least about 3.5 mm, such as at least about 3.7 mm, for example at least about 4 mm, such as at least about 4.5 mm, for example at least 5 mm, such as at least about 5.5 mm, for example at least about 6 mm. In an example where both suction channels 30, 32 have an inner diameter of 6 mm, the combined cross-sectional area of the suction channels 30, 32 would be about 56.5 mm², which is about 818% larger than that of the example conventional gastroscope (about 6.2 mm) and about 426%% larger than that of the example therapeutic gastroscope (about 10.8 mm²). And since the suction capacity is proportional to the combined cross-sectional area of the openings of the suction channels 230, 232, this results in substantially higher suction capability for the endoscope device 210 of the present disclosure as compared to the more conventional gastroscopes. In an example, the suction channels 230, 232 have a combined internal cross-sectional area of at least about 15 square millimeters (mm²), such as at least about 16 mm², for example at least about 16.5 mm², such as at least about 17 mm², for example at least about 17.5 mm², such as at least about 18 mm², for example at least about 18.5 mm², such as at least about 19 mm², for example at least about 19.5 mm², such as at least about 19.6 mm², for example at least about 20 mm², such as at least about 20.5 mm², for example at least about 21 mm², such as at least about 22 mm², for example at least about 23 mm², such as at least about 24 mm², for example at least about 25 mm², such as at least about 30 mm², for example at least about 35 mm², such as at least about 37.5 mm², for example at least about 40 mm², such as at least about 42.5 mm², for example at least about 45 mm², such as at least about 47.5 mm², for example at least about 50 mm², such as at least about 52.5 mm², for example at least about 55 mm², such as at least about 56 mm², for example at least about 56.5 mm², such as at least about 57.5 mm², for example at least about 60 mm².

In an example, the suction channels 230, 232 are coupled at least to the endoscope shaft 224, such as the shaft of a low-profile endoscope (such as Olympus EVIS EXERA III GIF-XP190N). In the example shown in FIGS. 9 and 10, the suction channels 230, 232 are coupled to the endoscope shaft 224 with one or more coupling devices or structures, such as medical tape 238 that couples the suction channels 230, 232 to the endoscope shaft 224 at one or more locations along the shaft 224, such as at 3 or more locations along the shaft 24. However, those having skill in the art will appreciate that other means of coupling the suction channels 230, 232 to the endoscope shaft 224 can be used without varying from the scope of the present disclosure, including, but not limited to clips, straps, adhesive, plastic welding.

In an example, at least one connection between the suction channels 230, 232 and the endoscope shaft 224, e.g., with the tape 238 or another coupling structure, is located proximate to the distal tip 222 of the shaft 224 and the distal ends 234, 236 of the suction channels 230, 232 so that when the distal tip 222 of the shaft 224 is deflected while maneuvering the insertion member 214, the distal ends 234, 236 of the suction channels 230, 232 will also be deflected in the same direction and with approximately the same amount of deflection. The matching deflection of the suction channels 230, 232 with that of the endoscope shaft 224 can ensure that direction of suction into the suction channels 230, 232 is pointed generally or substantially in the same direction as the digital imaging device 226 so that the suction will clear the visual field of the digital imaging device 226. In an example, the suction channels 230, 232 are coupled closely to the shaft 224 so that the shaft 224 and the suction channels 230, 232 can be more easily inserted into the patient cavity, e.g., so that the endoscope shaft 224 and the suction channels 230, 232 together act as the insertion member 214 of the endoscope device 210.

Suction can be supplied to the suction channels 230, 232 by a suction source device 240 via one or more suction supply lines 242. In an example, the suction channels 230, 232 and the one or more suction supply lines 242 can be configured to receive suction energy from a suction source device 240 comprising any of the many devices used in the medical field for providing suction. In an example, the suction source device 240 is a standard wall suction, e.g., that is supplied to operating theaters in most hospitals. In another example, the suction source device 240 can be an enhanced suction device, which may or may not be portable, that is configured to supply a larger suction force compared to standard wall suction devices. An example of such an enhanced suction device that can be used as the suction source device 240 includes, but is not limited to, the system sold under the NEPTUNE 3 Waste Management System trade name by Stryker Corp., Kalamazoo, MI, USA. In an example, the one or more suction supply lines 42 are separate from the umbilical cable 228, as shown in FIG. 9.

In an example, suction through the suction channels 230, 232 is controllable, either controllable for the suction channels 230, 232 collectively or for each suction channel 230, 232 individually. For example, the user of the endoscope device 210 may be able to turn on and turn off suction through one or both of the suction channels 230, 232. In other examples, the amount of suction force that is drawn through one or both of the suction channels 230, 232 can be controlled, such as with the one or more controls 216 or with some other controlling mechanism.

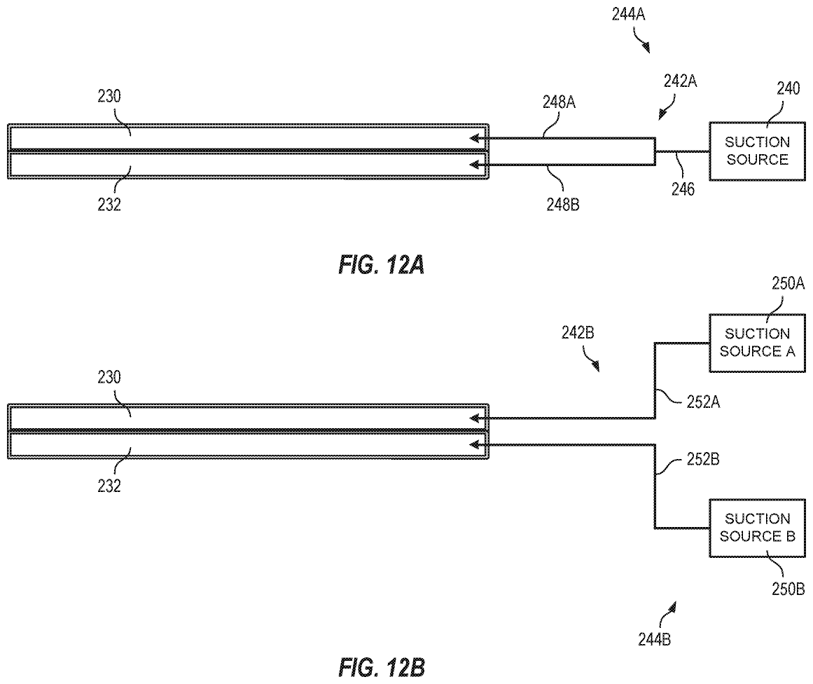

FIGS. 12A and 12B show schematic diagrams of two example suction supply systems 244A and 244B for supplying suction energy to the two suction channels 230, 232 of the endoscope device 210 of FIGS. 9-11. In a first example suction supply system 244A of FIG. 12A, a common suction source device 240 supplies suction energy to both the first suction channel 230 and the second suction channel 232 through a first example subsystem of suction supply lines 242A. In an example, the suction supply lines 242A include a common primary supply line 246 that is in fluid communication with the common suction source device 240 and that splits into a pair of separate supply branches 248A and 248B that are each in fluid communication with the primary supply line 246. Each supply branch 248A, 248B supplies suction energy to a separate suction channel 230, 232, e.g., with the first supply branch 248A being in fluid communication with and supplying suction energy to the first suction channel 230 and the second supply branch 248B being in fluid communication with and supplying suction energy to the second suction channel 232.

In an example, each suction supply branch 248A, 248B comprises the same tube that defines the corresponding suction channel 230, 232 to which the suction supply branch 248A, 248B supplies suction energy. In other words, in an example, the first supply branch 248A and the first suction channel are the same structure and the second supply branch 248B and the second suction channel 232 are the same structure. However, those having ordinary skill in the art will be able to readily design the suction supply system 244A where each supply branches 248A, 248B is a separate from its corresponding suction channel 230, 232 that can be connected in some way so the supply branch 248A, 248B and its corresponding suction channel 230, 232 are in fluid communication.

The first suction supply system 244A of FIG. 12A is a simplified design that requires only the single common suction source device 240 and the branched suction supply lines 242A. However, the first suction supply system 244A results in the suction energy supplied by the single common suction source device 40 being divided between the separate suction channels 230, 232, which would result in the suction energy that is supplied to each suction channel 230, 232 being weakened compared to the total suction energy the suction source device 240 can deliver. The single suction source device 240 may also make it more difficult to separately control the suction through each suction channel 230, 232.

FIG. 12B shows a second example suction supply system 244B that includes a separate suction source device 250A, 250B for each of the suction channels 230, 232, e.g., a first suction source device 250A that supplies suction energy to the first suction channel 230 and a second suction source device 250B that supplies suction energy to the second suction channel 232. The suction energy is supplied to the suction channels 230, 232 by their corresponding suction source devices 250A, 250B via a second example subsystem of suction supply lines 242B. In an example, the suction supply lines 242B includes separate dedicated suction supply lines 252A and 252B for each of the suction channels 230, 232. A first suction supply line 252A is in fluid communication with the first suction source device 250A at one end and with the first suction channel 230 at an opposite end in order to supply suction energy to the first suction channel 230 from the first suction source device 250A. A second suction supply line 252B is in fluid communication with the second suction source device 250B at one end and with the second suction channel 232 at an opposite end to supply suction energy to the second suction channel 232 from the second suction source device 250B.

In an example, each suction supply line 252, 252B comprises the same tube that defines the corresponding suction channel 230, 232 to which the suction supply line 252A, 252B supplies suction energy. In other words, in an example, the first supply line 252A and the first suction channel are the same structure and the second supply line 252B and the second suction channel 232 are the same structure. However, those having ordinary skill in the art will be able to readily design the suction supply system 244B where each of the supply lines 252A, 252B are a separate structure from its corresponding suction channel 230, 232 that can be connected in some way so the supply line 252A, 252B and its corresponding suction channel 230, 232 are in fluid communication.

The second suction supply system 244B adds more complexity and expense compared to the first suction supply system 244A because of the additional suction source device 250B and potentially additional control mechanisms to separately control suction energy from two suction source devices 250A, 250B through two separate suction supply lines 252A, 252B to the two separate suction channels 230, 232. However, the second suction supply system 244B also allows for more assurance that the suction energy supplied to each suction channel 230, 232 is at the desired intensity because the suction energy from each source device 250A, 250B is not being divided. Also, the second supply system 244B can provide for more control over the suction energy supplied to each suction channel 230, 232, including allowing different suction power to be supplied to each suction channel 230, 232 if the procedure being performed called for it.

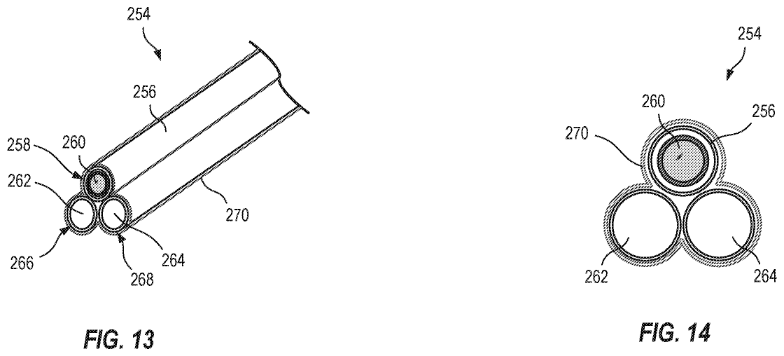

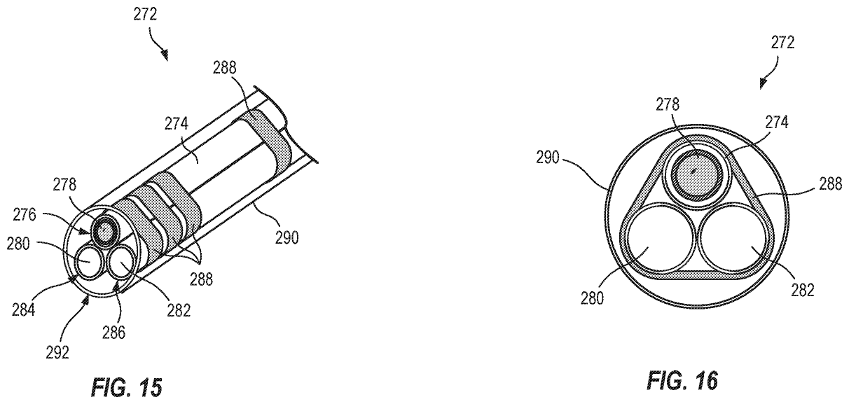

FIGS. 13-16 show various views of some alternative insertion members 254, 272 that can be used with an endoscope device similar to the endoscope device 210 described above with respect to FIGS. 9-11. FIGS. 13 and 14 show a perspective and end view, respectively, of a first alternative example insertion member 254 and FIGS. 15 and 16 show perspective and end views, respectively, of a second alternative example insertion member 272. The insertion members 254, 272 of FIGS. 13-16 can each be used with an endoscope device for examination of a cavity within a patient, such as a gastroscope. The endoscope device with which the insertion member 254 or the insertion member 272 can be used can include many of the same structures as the endoscope device 210 described above with respect to FIGS. 9-11. For example, the endoscope device used with the insertion member 254 or the insertion member 272 can include: an operating unit similar to the operating unit 212 to which the insertion member 254, 272 is coupled; one or more controls similar to the controls 216 so that a user can operate the endoscope device; a tool inlet channel similar to the tool inlet channel 218; and an umbilical cable similar to the umbilical cable 228.

The insertion member 254 of FIGS. 13 and 14 includes an elongated endoscope shaft 256 similar to the endoscope shaft 224 of endoscope device 210, which extends between a proximal end at the operating unit and a distal tip 258, a digital imaging device 260 coupled to the 5 distal tip 258 of the endoscope shaft 256, and a pair of elongated suction channels 262, 264 that each extend in generally the same direction as the endoscope shaft 256. The suction channels 262, 264 can be similar or identical to the suction channels 230, 232 of endoscope device 210, e.g., with the suction channels 262, 264 being positioned adjacent to the endoscope shaft 256 so that the shaft 256 and the suction channels 262, 264 are effectively running parallel to one another. The first suction channel 262 extends to a distal end 266 and the second suction channel 264 extends to a distal end 268. The suction channels 262, 264 are positioned so that the distal ends 266, 268 are located proximate to the distal tip 258 of the endoscope shaft 256 so that suction that is provided through the suction channels 262, 264 can clear away debris, such as blood, blood clots, food particles, and residual gastric content, so that the debris is clear of the visual field of the digital imaging device 258 at the distal tip 258.

The primary difference between the insertion member 254 of FIGS. 13 and 14 and the insertion member 214 for the endoscope device 210 of FIGS. 9-11 is the means of coupling the suction channels 262, 264 to the endoscope shaft 256. As mentioned above, in the example endoscope device 210 of FIGS. 9-11, the suction channels 230, 232 are coupled to the endoscope shaft 224 with one coupling devices or structures such as the medical tape 238 described above. In an example, the suction channels 262, 264 are coupled to the endoscope shaft 256 with a sheath 270 that contains the endoscope shaft 256 and the suction channels 262, 264. The sheath 270 can be a close fitting structure such that the suction channels 262, 264 are held in close proximity to the endoscope shaft 256, at least with the distal ends 266, 268 of the suction channels 262, 264 being held in close proximity to the distal tip 258 of the endoscope shaft 256.

In an example, the sheath 270 couples the suction channels 262, 264 to the endoscope shaft 256 so that when the distal tip 258 of the shaft 256 is deflected while maneuvering the insertion member 254, the distal ends 266, 268 of the suction channels 262, 264 will also be deflected in the same direction and with approximately the same amount of deflection. The matching deflection of the suction channels 262, 264 with that of the endoscope shaft 256 can ensure that direction of suction into the suction channels 262, 264 is pointed generally or substantially in the same direction as the digital imaging device 260 so that the suction will clear the visual field of the digital imaging device 260. In an example, the sheath 270 holds the suction channels 262, 264 closely to the endoscope shaft 256 so that the shaft 256 and the suction channels 262, 264 can be more easily inserted into the patient cavity. In an example, the sheath 270 is made from a resilient material so that it will be unlikely to damage tissue of the patient if the sheath 270 comes into contact with the tissue.

Turning to FIGS. 15 and 16, the insertion member 272 includes an elongated endoscope shaft 274 similar to the endoscope shafts 224 and 256, which extends between a proximal end at the operating unit and a distal tip 276, a digital imaging device 278 coupled to the distal tip 276 of the endoscope shaft 274, and a pair of elongated suction channels 280, 282 that each extend in generally the same direction as the endoscope shaft 274. The suction channels 280, 282 can be similar or identical to the suction channels 230, 232 of endoscope device 210 and to the suction channels 262, 264 of the insertion member 254, e.g., with the suction channels 280, 282 being positioned adjacent to the endoscope shaft 274 so that the shaft 274 and the suction channels 280, 282 are effectively running parallel to one another. The first suction channel 280 extends to a distal end 284 and the second suction channel 282 extends to a distal end 286. The suction channels 280, 282 are positioned so that the distal ends 284, 286 are located proximate to the distal tip 276 of the endoscope shaft 274 so that suction provided through the suction channels 280, 282 can clear away debris, such as blood, blood clots, food particles, and residual gastric content, so that the debris is clear of the visual field of the digital imaging device 278 at the distal tip 276. In an example, the suction channels 280, 282 are coupled to the endoscope shaft 74 with medical tape 288 or another coupling mechanism or structure, at least proximate to the distal tip 276 so that, in an example, the distal ends 284, 286 of the suction channels 280, 282 can deflect with the distal tip 276 of the endoscope shaft 274.

In addition to these aspects, the insertion member 274 in FIGS. 15 and 16 can include an optional overtube 290 through which the endoscope shaft 274 and the suction channels 280, 282 can be inserted. In an example, the overtube 290 can make insertion of the insertion member 272 into the patient cavity easier, e.g., because the outer surface of the overtube 290 is smoother than the outside profile of the endoscope shaft 274, the suction channels 280, 282, and the tape 288. The overtube 290 can have a size that is large enough to cover both the endoscope shaft 274 and the suction channels 280, 282, but not so large that it cannot be inserted into the patient cavity of interested. In an example, the overtube 290 has an inner diameter of from about 10 mm to about 20 mm, such as from about 15 mm to about 17.5 mm, for example about 16.7 mm. In an example, the overtube 290 is a standard overtube, such as those sold under the GUARDUS trademark by Steris Endoscopy, Mentor, OH, USA.

The overtube 290 can be configured so that the distal tip 276 of the endoscope shaft 274 and the distal ends 284, 286 of the suction channels 280, 282 can be extended out beyond a distal end 292 of the overtube 290. The overtube 290 can also be configured so that the endoscope shaft 274 and the suction channels 280, 282 can be retracted inside of the overtube 290, e.g., so that the distal tip 276 of the endoscope shaft 274 and the distal ends 284, 286 of the suction channels 280, 282 are behind the distal end 292 of the overtube 290 within a lumen 294 of the overtube 290 during insertion into the patient cavity. Once the distal end 292 of the overtube 290 reaches the target site, the endoscope shaft 274 and the suction channels 280, 282 can be extended out past the distal end 292 of the overtube 290 to reach the target site.

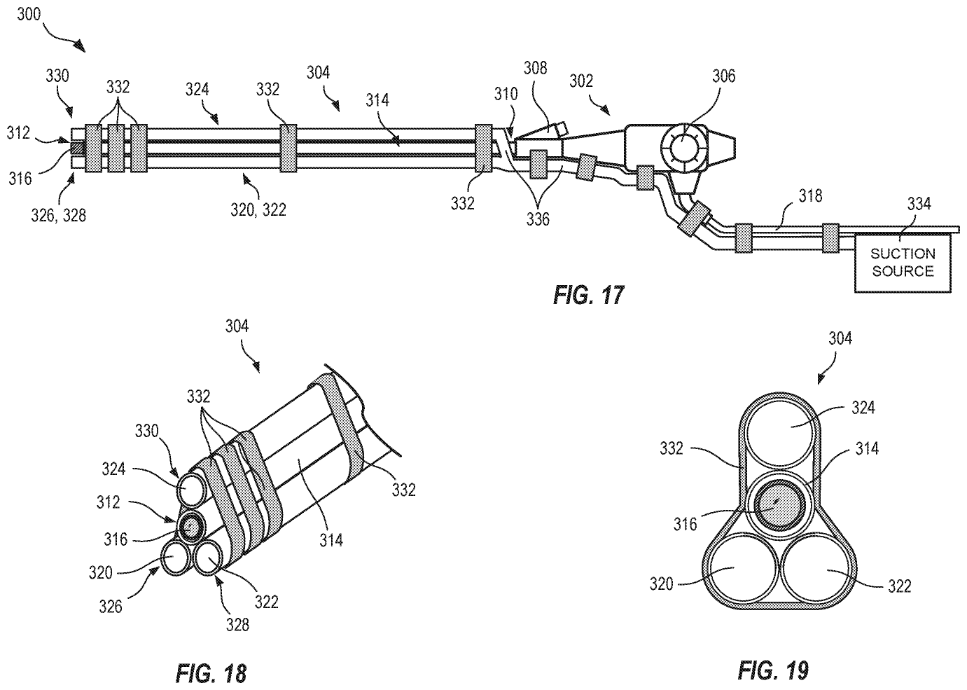

The present disclosure is not limited to an endoscope device or endoscope accessory with only two suction channels, such as is shown in FIGS. 9-11 and 13-16. Rather, an endoscope device or accessory could include three or more suction channels that are positioned proximate to the endoscope shaft of the endoscope. FIG. 17 shows an example of just such an endoscope device 300 that, like the endoscope device 210 of FIGS. 9-11, can be used for examination of a cavity within a patient, such as a gastroscope. The endoscope device 300 in FIG. 17 includes many of the same structures as the endoscope device 210 of FIG. 1, which can be similar or even identical to the structures described above with respect to the endoscope device 210. For example, the endoscope device 300 can include: an operating unit 302; an insertion member 304 coupled to the operating unit 302; one or more controls 306; a tool inlet channel 308; an elongated endoscope shaft 314 that extends between a proximal end 310 coupled to the operating unit 302 and a distal tip 312; a digital imaging device 316 at the distal tip 312 of the shaft 314; and an umbilical cable 318 coupled to the operating unit 302 to provide specified functionality to the endoscope device 300.

The example endoscope device 300 of FIG. 17 includes a set of elongated suction channels 320, 322, 324, which serve the same purpose as that of the suction channels 230, 232 in the endoscope device 210, i.e., to provide suction at of proximate to the distal tip 312 of the endoscope shaft 314 in order to clear debris commonly associated with gastrointestinal endoscopy, such as food particles, blood, blood clots, and other residual gastric content. However, instead of a pair of suction channels such as the suction channels 230, 232 of the endoscope device 210, the endoscope device 300 includes three suction channels 320, 322, 324 that are dispersed around the endoscope shaft 314. FIG. 18 shows a perspective view of the insertion member portion 304 of the endoscope device 300 and FIG. 19 shows an end view of the distal end of the insert member 304.

In an example, a first of the suction channels, e.g., the suction channel 320, and a second of the suction channels, e.g., the suction channel 322, are located below the endoscope shaft 314, e.g., spaced laterally on opposing sides of the endoscope shaft 314, while a third of the suction channels, e.g., the suction channel 324, is positioned above the endoscope shaft 314. In an example, the three suction channels 320, 322, and 324 are evenly or substantially evenly spaced radially around the endoscope shaft 314 so that the suction through the suction channels 320, 322, 324 will be distributed evenly or substantially evenly around the distal tip 312 of the endoscope shaft 314 in order to evenly clear the visual field of the digital display device 316. For example, with the three suction channels 320, 322, 320 shown in FIGS. 17-19, each suction channel 320, 322, 324 can be spaced approximately 120° from the other two suction channels 320, 322, 324 such that this even spacing is achieved. In examples of other numbers of suction channels, the spacing between adjacent suction channels can be different. For example, if the endoscope device includes four suction channels, then each of the four suction channels can be spaced approximate 90° from two adjacent suction channels so that the four suction channels are evenly or substantially evenly spaced around the endoscope shaft.

In an example, the first suction channel 320 extends to a distal end 326, the second suction channel 322 extends to a distal end 328, and the third suction channel 324 extends to a distal end 330. Similar to the suction channels 230 and 232 of the endoscope device 210, the suction channels 320, 322, and 324 of the endoscope device 300 can run parallel to the endoscope shaft 314, e.g., so that the distal ends 326, 328, 330 of the suction channels 320, 322, 324 will be aligned in substantially the same direction as the distal tip 312 of the endoscope shaft 314.

In an example, the suction channels 320, 322, 324 have a combined internal cross-sectional area of at least about 15 mm², such as at least about 16 mm², for example at least about 16.5 mm², such as at least about 17 mm), for example at least about 17.5 mm², such as at least about 18 mm², for example at least about 18.5 mm², such as at least about 19 mm², for example at least about 19.5 mm², such as at least about 19.6 mm², for example at least about 20 mm², such as at least about 20.5 mm², for example at least about 21 mm², such as at least about 22 mm², for example at least about 23 mm², such as at least about 24 mm², for example at least about 25 mm² such as at least about 30 mm², for example at least about 35 mm², such as at least about 37.5 mm², for example at least about 40 mm², such as at least about 42.5 mm², for example at least about 45 mm², such as at least about 47.5 mm², for example at least about 50 mm², such as at least about 52.5 mm², for example at least about 55 mm², such as at least about 56 mm², for example at least about 56.5 mm², such as at least about 57.5 mm², for example at least about 60 mm².

In an example, the suction channels 320, 322, 324 are coupled to the endoscope shaft 314 with one or more coupling devices or structures, such as medical tape 332 that couples the suction channels 320, 322, 324 to the endoscope shaft 314 at one or more locations along the shaft 314. In an example, at least one connection between the suction channels 320, 322, 324 and the endoscope shaft 314, e.g., with the tape 332 or another coupling structure being located proximate to the distal tip 312 of the shaft 314 and the distal ends 326, 328, 330 of the suction channels 320, 322, 324 so that when the distal tip 312 of the shaft 314 is deflected while maneuvering the insertion member 304, the distal ends 326, 328, 330 of the suction channels 320, 322, 324 will also be deflected in the same direction and with approximately the same amount of deflection. The matching deflection of the suction channels 320, 322, 324 with that of the endoscope shaft 314 can ensure that direction of suction into the suction channels 320, 322, 324 is pointed generally or substantially in the same direction as the digital imaging device 316 so that the suction will clear the visual field of the digital imaging device 316.

Similar to the endoscope device 210, suction can be supplied to the suction channels 320, 322, 324 of the endoscope device 300 from one or more suction source devices 334 via one or more suction supply lines 336. The one or more suction source devices 334 and the one or more suction supply lines 336 can be similar or identical to the suction source 240 and the one or more suction supply lines 242 described above with respect to the endoscope device 210.

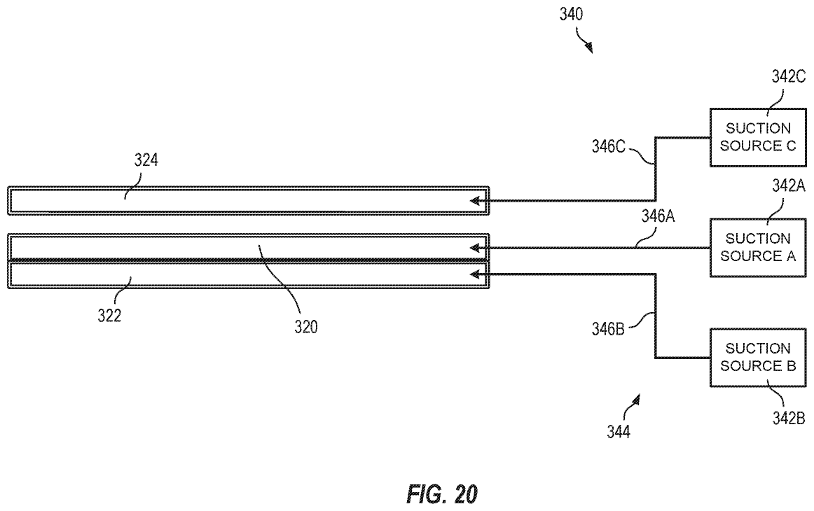

FIG. 20 shows a schematic diagram of an example suction supply system 340 for supplying suction energy to the three suction channels 320, 322, 324 of the endoscope device 300 of FIG. 17. In an example, the suction supply system 340 includes a separate suction source device for each of the suction channels 320, 322, 324, e.g., a first suction source device 342A that supplies suction energy to the first suction channel 320, a second suction source device 342B that supplies suction energy to the second suction channel 322, and a third suction source device 342C that supplies suction energy to the third suction channel 324.

The suction energy is supplied to the suction channels 320, 322, 324 by their corresponding suction source devices 342A, 342B, 342C via a subsystem of suction supply lines 344. In an example, the suction supply subsystem 344 includes separate a dedicated suction supply line 346A, 346B, and 346C for each of the suction channels 320, 322, 324. In an example, a first suction supply line 346A is in fluid communication with the first suction source device 3425A at one end and with the first suction channel 320 at an opposite end in order to supply suction energy to the first suction channel 320 from the first suction source device 342A. A second suction supply line 346B is in fluid communication with the second suction source device 342B at one end and with the second suction channel 322 at an opposite end to supply suction energy to the second suction channel 322 from the second suction source device 346B. A third suction supply line 346C is in fluid communication with the third suction source device 342C at one end and with the third suction channel 324 at an opposite end to supply suction energy to the third suction channel 324 from the third suction source device 346C.

In an example, each suction supply line 346A, 346B, 346C comprises the same tube or structure that defines the corresponding suction channel 320, 322, 324 to which the suction supply line 346A, 346B, 346C supplies suction energy. In other words, in an example, the first supply line 346A and the first suction channel 320 are the same structure, e.g., the same first tube, the second supply line 346B and the second suction channel 322 are the same structure, e.g., the same second tube, and the third supply line 346C and the third suction channel 324 are the same structure, e.g., the same third tube. However, those having ordinary skill in the art will be able to readily design the suction supply system 340 so that each of the supply lines 346A, 346B, 346C are a separate structure from its corresponding suction channel 320, 322, 324 that can be connected in some way so the supply line 346A, 346B, 346C and its corresponding suction channel 320, 322, 324 are in fluid communication.

In another example, suction energy can be supplied to all three suction channels 320, 322, 324 from a common suction source device, similar to the suction supply system 244A described above with respect to FIG. 12A for the suction channels 230, 232 of the endoscope device 210. In yet another example (not shown), two of the three suction channels can be supplied by a first common suction source device, while the third suction channel can be supplied by its own dedicated suction source device. For example, the first and second suction channels 320 and 320 can be supplied by a first suction source device with supply branches (similar to the supply branches 248A, 248B shown in FIG. 12A) and the third suction channel 324 can be supplied by a second suction source device with its own dedicated suction supply line.

[symbol] In one example, any of the components that make up any one of the example endoscope devices 10, 40, 80, 120, 210, 300 can be made from disposable material (e.g., so that once a component has been in contact with a patient, it can be disposed of). For example, the structures that form any one of the endoscope shafts 24, 54, 94, 134, 224, 256, 274, and 314 or any one of the suction channels 30, 60, 100, 140, 230, 232, 262, 264, 280, 282, 320, 322, and 324 can be made from disposable material. Alternatively, one or more of the components can be made from reusable materials, preferably reusable materials that can be easily sterilized.

Although each of the endoscope devices 10, 40, 80, 120, 210, and 300 are described as being configured for examining the gastrointestinal tract, e.g., as a gastroscope, those having ordinary skill in the art will appreciate that the features of the endoscope devices 10, 40, 80, 120, 210, and 300 can be configured for use in other cavities or lumens within the body for any procedure where endoscopically guided suction would be helpful or required, including, but not limited to: examination of the tracheo-bronchial tree, e.g., during bronchoscopy; examination of the colon, e.g., for a colonoscopy; fluid collection; or a necrosis removal procedure, such as the removal of pancreatic walled off necrosis.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An endoscope device comprising:
an operating unit comprising one or more controls for operating the endoscope device; and
an elongate insertion member coupled to the operating unit, the elongate insertion member comprising:
an elongate endoscope shaft extending from a shaft proximal end proximate to the operating unit to a shaft distal end;
a digital imaging device coupled to the shaft distal end of the elongate endoscope shaft; and
an elongate suction channel with a channel distal end that is positioned proximate to the shaft distal end so that suction applied to the elongate suction channel can clear debris from a visual field of the digital imaging device.

2. An endoscope device according to claim 1, wherein an outlet opening of the elongate suction channel has a cross-sectional area of at least about 15 square millimeters.

3. (canceled)

4. (canceled)

5. An endoscope device according to claim 1, wherein the elongate suction channel is a tubular member positioned adjacent to the elongate endoscope shaft and that extends parallel or substantially parallel to the elongate endoscope shaft.

6. An endoscope device according to claim 1, wherein the elongate suction channel is a sheath positioned around the elongate endoscope shaft so that there is an annular space between an outer surface of the endoscope shaft and an inner surface of the elongate suction channel.

7. An endoscope device according to claim 1, further comprising an overtube surrounding at least a portion of the elongate endoscope shaft and the elongate suction channel.

8. (canceled)

9. An endoscope device according to claim 1, wherein the elongate endoscope shaft has an outer size of 9 mm or less.

10. (canceled)

11. (canceled)

12. An accessory for an endoscope, the accessory comprising:
an elongate suction channel configured for coupling to an elongated endoscope shaft of an insertion member of the endoscope, wherein the elongate suction channel is configured so that when the elongate suction channel is coupled to the elongate endoscope shaft, a channel distal end of the elongate suction channel will be positioned proximate to a shaft distal end of the elongate endoscope shaft so that suction applied to the elongate suction channel can clear debris from an area around the shaft distal end.

13. An accessory according to claim 12, wherein an outlet opening of the elongate suction channel has a cross-sectional area of at least about 15 square millimeters.

14. (canceled)

15. (canceled)

16. An accessory according to claim 12,
wherein the elongate suction channel is a tubular member configured to be positioned adjacent to the elongate endoscope shaft such that the elongate suction channel extends parallel or substantially parallel to the elongate endoscope shaft.

17. An accessory according to claim 12,
wherein the elongate suction channel is a sheath configured to be positioned around the elongate endoscope shaft so that there is an annular space between an outer surface of the elongate endoscope shaft and an inner surface of the elongate suction channel.

18. An accessory according to claim 12, further comprising an overtube configured to surround at least a portion of the elongate endoscope shaft and elongate the suction channel.

19. An endoscope device comprising:
an operating unit comprising one or more controls for operating the endoscope device; and
an elongate insertion member coupled to the operating unit, the elongate insertion member comprising:
an elongate endoscope shaft extending from a shaft proximal end proximate to the operating unit to a shaft distal end;
a digital imaging device coupled to the shaft distal end of the elongate endoscope shaft; and
a plurality of elongate suction channels each comprising a channel distal end that is positioned proximate to the shaft distal end so that suction applied to the plurality of elongate suction channels can clear debris from a visual field of the digital imaging device.

20. An endoscope device according to claim 19, wherein outlet openings of the plurality of elongate suction channels have a combined cross-sectional area of at least about 15 square millimeters.

21-23. (canceled)

24. An endoscope device according to claim 19, wherein each of the plurality of elongate suction channels comprises a tubular member positioned adjacent to the elongate endoscope shaft and that extends parallel or substantially parallel to the elongate endoscope shaft.

25. An endoscope device according to claim 19, wherein the elongate insertion member further comprises one or more coupling structures or mechanisms that couple the plurality of elongate suction channels to the elongate endoscope shaft.

26. (canceled)

27. An endoscope device according to claim 19, further comprising an overtube surrounding at least a portion of the elongate endoscope shaft and the plurality of elongate suction channels.

28. (canceled)

29. An endoscope device according to claim 19, wherein the elongate endoscope shaft has an outer size of 9 mm or less.

30-32. (canceled)

33. An accessory according to claim 41, wherein outlet openings of the elongate suction channel and the one or more additional elongate suction channels have a combined cross-sectional area of at least about 15 square millimeters.

34-36. (canceled)

37. An accessory according to claim 41, wherein each of the one or more additional elongate suction channels comprises a tubular member configured to be positioned adjacent to the elongate endoscope shaft such that each of the one or more additional elongate suction channels extends parallel or substantially parallel to the elongate endoscope shaft.

38-40. (canceled)

41. An accessory according to claim 12, further comprising one or more additional elongate suction channels comprising a second channel distal end configured for coupling to the elongate endoscope shaft of the insertion member so that the second channel distal end of each of the one or more additional elongate suction channels is positioned proximate to the shaft distal end, wherein the elongate suction channel and the one or more additional elongate suction channels are configured so that suction applied to the elongate suction channel and to the one or more additional elongate suction channels will clear debris from the area around the shaft distal end.
